(19) United States
(12) Patent Application Publication
(10) Pub. No.: US 2014/0186275 A1
(43) Pub. Date: Jul. 3, 2014

(54) TOOTHPASTE FOR ELECTRIC TOOTHBRUSHES

(71) Applicant: HENKEL AG & CO. KGGA, Dusseldorf (DE)

(72) Inventors: Christel Adomat, Dusseldorf (DE); Nicole Duschek, Dusseldorf (DE); Claudia Hundeiker, Meerbusch (DE)

(73) Assignee: HENKEL AG & CO. KGGA, Dusseldorf (DE)

(21) Appl. No.: 14/199,340

(22) Filed: Mar. 6, 2014

Related U.S. Application Data
(63) Continuation of application No. PCT/EP2012/065938, filed on Aug. 15, 2012.

(30) Foreign Application Priority Data
Sep. 9, 2011 (DE) 102011082433.2

(51) Int. Cl. A61K 8/46; A61Q 11/00; A61K 8/44

(52) U.S. Cl. CPC A61K 8/463; A61K 8/442; A61Q 11/00
USPC 424/54

(57) ABSTRACT

Agents and methods are provided for toothpaste for an electric toothbrush. The oral and dental care and cleaning agent includes at least one anionic surfactant and at least one amphoteric surfactant. A weight ratio of the at least one anionic surfactant to the at least one amphoteric surfactant(s) is ≧6.

### RELATED APPLICATIONS

The present specification is a U.S. continuation patent application under 35 U.S.C. 111(a) and claims the right of priority under 35 U.S.C. 365 to international patent Application No. PCT/EP2012/065938, filed Aug. 15, 2012, entitled “SPECIAL TOOTHPASTE FOR ELECTRIC TOOTH BRUSHES II” which claims benefit of German application No.: 102011082433.2, filed Sep. 9, 2011, these applications are herein incorporated by reference in their entirety.

### FIELD OF THE INVENTION

The present invention generally relates to a dentifrice (i.e., a toothpaste) that takes into account the particular requirements associated with the use of electric toothbrushes.

### BACKGROUND OF THE INVENTION

Dental cleaning agents are on the market in a variety of forms, and are used to clean the tooth surface and to prevent diseases of the teeth and gums. They may contain a combination of polishing agents, humectants, surfactants, binding agents, flavorings, and fluoride-containing and antimicrobial active agents. Besides tooth powders, which play a subordinate role because of their elevated abrasiveness, dental cleaning agents are offered principally in the form of a paste, cream, translucent gel, or transparent gel. Liquid dentifrices and mouthwashes have also become increasingly significant in recent years.

Developments have also been pursued in terms of equipment. Besides the conventional manual toothbrush, which the consumer moves in the mouth using circular motions; electric toothbrushes have also become established on the market. Electric toothbrushes generate some of the motion of the bristles on the tooth surface by means of a battery (usually rechargeable) in a hand piece of the toothbrush. Accordingly, the consumer may, depending on the model, replace the circular hand motion with a horizontal linear motion.

Electric toothbrushes are common on the market in a variety of configurations. For example, round brush heads are electrically rotated or partly rotated, with clockwise and counterclockwise rotations. Elongated brush heads modeled on conventional toothbrushes may be electrically oscillated.

Electric toothbrushes may relieve the consumer of some of the specific motion sequences used to clean the teeth. These advantages are also accompanied, however, by disadvantages. For example, an electric toothbrush usually exerts a more intense load on the tooth surface than in the case of manual tooth brushing. If the nature and concentration of the abrasive materials in the dentifrice are not coordinated with this load, damage to the tooth enamel may occur in connection with electric toothbrush use.

A further problem is the fact that the complex mechanism in the toothbrush may become clogged, and the toothbrush may not allow movement of the brush head. Additional attention is also given to rheology, since a dentifrice should adhere to the electric toothbrush head in order to prevent the dentifrice from spraying.

Some of the aforementioned requirements are mutually contradictory, since abrasive materials are at least in part also consistency agents.

Accordingly, the present systems and methods provide an oral and dental care and cleaning agent that is suitable specifically for electric toothbrushes and that overcomes the disadvantages recited above. More specifically, the present systems and methods achieve a high level of cleaning performance without excessive abrasiveness. The present systems and methods also allow for low stress on the sensitive mechanism of the electrically operated brush heads and sufficient adhesion to the brush head.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An oral and dental care and cleaning agent containing at least one anionic surfactant and at least one amphoteric surfactant, characterized in that the weight ratio of anionic surfactant to amphoteric surfactant is greater than or equal to 6.

### BRIEF DESCRIPTION OF THE DRAWINGS

The present invention will hereinafter be described in conjunction with the following drawing figures, wherein like numerals denote like elements, and

FIG. 1 is an image of a brush head prepared with Formula V1;

FIG. 2 is an image of a brush head prepared with Formula V2; and

FIG. 3 is an image of a brush head prepared with Formula E.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

According to the present specification, anionic surfactants and amphoteric surfactants may be used with differing ratio with respect to one another in order to overcome the disadvantages mentioned above. In a first example, the present specification describes an oral and dental care and cleaning agent comprising: at least one anionic surfactant, and at least one amphoteric surfactant. The weight ratio of anionic to amphoteric surfactant(s) is ≧6 The quantities used are preferably also selected within narrow limits.

As used in the present specification and in the appended claims, the phrases “oral and dental care agents,” “oral and dental cleaning agents,” “oral care agents,” “dental care agents” or similar terminology refer to oral and dental powders, oral and dental pastes, liquid oral and dental creams, oral and dental rinses, and oral and dental gels. Toothpastes and liquid dental cleaning agents are preferably suitable. The oral and dental care and cleaning agents may be in the form of toothpastes, liquid dentifrices, tooth powders, mouthwashes, or a chewing compound, e.g. as chewing gum. Preferably, however, they are present as more or less flowable or plastic toothpastes, such as those used to clean teeth using a toothbrush. A further particularly preferred example of the present agent is an oral rinse solution and mouthwash that is used to rinse out the oral cavity.

As a first ingredient, an oral and dental care and cleaning agent may include an anionic surfactant(s). Preferred oral and dental care and cleaning agents may contain, based on their weight, 0.25 to 4 wt %, preferably 0.5 to 3.5 wt %, more preferably 0.75 to 3 wt %, even more preferably 1 to 2.5 wt %, and in particular 1.6 to 2.2 wt % anionic surfactant(s).

Examples of anionic surfactants may include soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, monoglyceride (ether)sulfates, fatty acid amide (ether)sulfates, mono- and dialkyl sulfosuccinates, mono- and dialkyl sulfosuccinamates, sulfotriglycerides, amide soaps, ethercarboxylic acids and salts thereof, fatty acid isethionates, fatty acid sarcosinates, fatty acid taurides, N-acylamino acids, for example acyl lactylates, acyl tartrates, acyl glutamates, and acyl aspartates, alkyl oligoglucoside sulfates, protein fatty acid condensates (in particular wheat-based plant products), alkyl (ether)phosphates, or combinations thereof. If the anionic surfactants contain polyglycol ether chains, they can exhibit a conventional but preferably a restricted homolog distribution.

Very particularly preferred oral and dental care and cleaning agents may also include an alkyl sulfate(s) as an anionic surfactant. More specifically, preferred oral and dental care and cleaning agents may include 0.25 to 4 wt %, preferably 0.5 to 3.5 wt %, more preferably 0.75 to 3 wt %, even more preferably 1 to 2.5 wt %, and in particular 1.6 to 2.2 wt % sodium dodecyl sulfate.

The oral and dental care and cleaning agents may include an amphoteric surfactant(s). “Amphoteric” surfactants and emulsifier agents may refer to those surface-active compounds that contain, besides a C₈ to C₂₄ alkyl or acyl group, at least one free amino group and at least one —COON or —SO₃H group, and are capable of forming internal salts. Examples of amphoteric surfactants may include N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Examples of preferred amphoteric surfactants include N-cocalkylaminopropionate, cocacylaminoethylaminopropionate, and C₁₂ to C₁₈ acyl sarcosine.

Particularly preferred oral and dental care and cleaning agents may include, based on their weight, 0.01 to 2 wt %, preferably 0.05 to 1.5 wt %, more preferably 0.1 to 1 wt %, even more preferably 0.12 to 0.7 wt %, and in particular 0.15 to 0.6 wt % amphoteric surfactant(s).

Particularly preferred oral and dental care and cleaning agents according to the present invention are characterized in that they contain amphoteric surfactant(s) from the groups of: N-alkylglycines, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids each having approximately 8 to 24 carbon atoms in the alkyl group, alkylaminoacetic acids each having approximately 8 to 24 carbon atoms in the alkyl group, N-cocalkylaminopropionate, cocacylaminoethylaminopropionate, C₁₂ to C₁₈ acyl sarcosine, N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, 2-alkyl-3-carboxymethyl-3-hydroxyethylimidazolines each having 8 to 18 carbon atoms in the alkyl or acyl group, cocacylaminoethylhydroxyethylcarboxymethyl glycinate, the compounds known by the INCI name Cocamidopropyl Betaine, the compounds known by the INCI name Disodium Cocamphodiacetate.

Particularly preferred oral and dental care and cleaning agents may include, as amphoteric surfactants, betaines of formula (Bet-I)

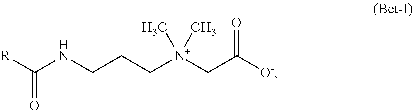

in which R denotes a straight-chain or branched, saturated or mono- or poly-unsaturated alkyl or alkenyl residue, respectively, having 8 to 24 carbon atoms.

These surfactants may be referred to according to INCI nomenclature as Amidopropyl Betaines; the representatives that derive from coconut fatty acids are preferred, and may be referred to as Cocamidopropyl Betaines. It is particularly preferred to use surfactants of formula (Bet-I) may be a mixture of the following representatives:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—CH₂)₃N⁺(CH₃)₂CH₂COO⁻.

Particularly preferably, surfactants of formula (Bet-I) are used within narrower quantitative ranges. Preferred in this context are oral and dental care and cleaning agents that contain, based on their weight, 0.01 to 2 wt %, preferably 0.05 to 1.5 wt %, more preferably 0.1 to 1 wt %, even more preferably 0.12 to 0.7 wt %, and in particular 0.15 to 0.6 wt % Cocamidopropyl Betaine.

In particularly preferred agents , the total quantity of surfactants is likewise limited. Oral and dental care and cleaning agents in which the total quantity of surfactants is 0.5 to 5 wt %, preferably 0.75 to 4.5 wt %, more preferably 1 to 4 wt %, even more preferably 1.25 to 3.5 wt %, and in particular 1.6 to 2.5 wt %, are preferred here.

In some examples, the weight ratio of anionic to amphoteric surfactant(s) is ≧6, i.e. the anionic surfactants are used at least at a sixfold (weight) excess in terms of the amphoteric surfactants. The “weight ratio” may refer to the weight ratio of all substances from the groups recited, or the active substances being calculated. For example, if an oral and dental care and cleaning agent contains 1 wt % pure sodium dodecyl sulfate and 0.4 wt % of a 25% surfactant solution of an amphoteric surfactant, the weight ratio is then 1/0.1=10.

Oral and dental care and cleaning agents preferred may exhibit a weight ratio of anionic to amphoteric surfactant(s) is ≧6.5, preferably ≧6.75, more preferably ≧7, even more preferably ≧7.5, particularly preferably ≧7.75, and in particular ≧8.

In oral and dental care and cleaning agents preferred, the weight ratio lies within an even narrower range. Particularly preferred oral and dental care and cleaning agents may be characterized in that the weight ratio of anionic to amphoteric surfactant(s) is in the range of ≧10.25 to ≦20, preferably in the range of ≧10.5 to ≦17.5, more preferably in the range of ≧10.75 to ≦15, even more preferably in the range of ≧11 to ≦12.5, and in particular in the range of ≧11.1 to ≦12.

The oral and dental care and cleaning agents preferably may contain at least one polishing agent. Polishing agents may refer to friction bodies used in a toothpaste, in particular those that contain no calcium ions. Preferably suitable polishing agent components may be silicic acids, aluminum hydroxide, aluminum oxide, sodium aluminum silicates, organic polymers, or mixtures of such friction bodies.

Calcium-containing polishing components, for example chalk, calcium pyrophosphate, dicalcium phosphate dihydrate may be contained in quantities of up to 5 wt %, based on the total composition.

The total polishing agent content may be preferably in the range from 5 to 50 wt % of the oral and dental care and cleaning agent. Oral and dental care and cleaning agents preferred contain polishing agents within narrower quantity ranges. Oral care agents preferred according to the present invention are characterized here in that they contain, based on their weight, 1 to 25 wt %, preferably 2.5 to 20 wt %, more preferably 5 to 18 wt %, and in particular 7.5 to 16 wt % polishing agent(s).

Toothpastes and liquid dental cleaning agents that contain silicic acids as polishing agent(s) are particularly preferred. Examples of silicic acids include silicic acid gels, silicic acid hydrogels, and precipitated silicic acids. Silicic acid gels may be manufactured by reacting sodium silicate solutions with strong aqueous mineral acids to form a hydrosol, aging to a hydrogel, washing, and drying. If drying occurs under mild conditions to water contents from 15 to 35 wt %, so-called silicic acid hydrogels may be obtained. Drying to water contents below 15 wt % results in an irreversible shrinkage of the previously loose structure of the hydrogel to the dense structure of the so-called xerogel.

A second preferably suitable group of silicic acid polishing agents is the precipitated silicic acids. These may be obtained by precipitating silicic acid from dilute alkali silicate solutions by adding strong acids under conditions in which aggregation to a sol and gel cannot occur. A precipitated silicic acid having a BET surface area from 15 to 110 m²/g, a particle size from 0.5 to 20 μm, where at least 80 wt % of the primary particles are to be below 5 μm, and a viscosity in a 30% glycerol/water dispersion (1:1) from 30 to 60 Pa.s (20° C.), is preferably suitable, in a quantity from 10 to 20 wt % of the toothpaste. Preferably suitable precipitated silicic acids of this kind may exhibit rounded corners and edges, and may be obtainable under the commercial name Sident® 12 DS (DEGUSSA).

Other precipitated silicic acids of this kind may be obtained under the commercial name Sident® 8 (DEGUSSA) and Sorbosil® AC 39 (Crosfield Chemicals). These silicic acids may include a reduced thickening effect and may have an average particle size of 8 to 14 μm with a specific surface area from 40 to 75 m²/g (according to BET), and are particularly suitable for liquid dentifrices. These may have a viscosity (25° C., shear rate D=10 s⁻¹) from 10 to 100 Pa.s.

On the other hand, toothpastes that have an appreciably higher viscosity of more than 100 Pa.s (25° C., D=10 s⁻¹) require a sufficiently high proportion of silicic acids having a particle size less than 5 μm, preferably at least 3 wt % of a silicic acid having a particle size from 1 to 3 μm. Even finer-particulate so-called “thickening” silicic acids having a BET surface area from 150 to 250 m²/g, for example the commercial products Sipernat 22 LS or Sipernat® 320 DS, may be added to such toothpastes in addition to the aforesaid precipitated silicic acids.

Further polishing-agent components that can be contained are also, for example, aluminum oxide in the form of low-calcined alumina having an α-and β-aluminum oxide content, in a quantity of approx. 1 to 5 wt %. A suitable aluminum oxide of this kind may be obtainable under the commercial name “ultrafine polishing alumina P10” (Giulini Chemie).

Also suitable as polishing agents may include friction bodies. Examples of friction bodies include sodium aluminum silicates such as e.g. zeolite A, organic polymers such as polymethacrylate, or combinations of these and the previously mentioned friction bodies.

Oral and dental care and cleaning agents according to the present specification may additionally contain cleaning bodies, preferably silicic acids, aluminum hydroxide, aluminum oxide, calcium pyrophosphate, chalk, dicalcium phosphate dihydrate (CaHPO₄.2H₂O), sodium aluminum silicates, in particular zeolite A, organic polymers, in particular polymethacrylates, or mixtures of said friction bodies, preferably in quantities from 1 to 30 wt %, preferably from 2.5 to 25 wt %, and in particular from 5 to 22 wt %, based in each case on the total agent, are preferred.

Oral and dental care and cleaning agents particularly preferred may contain exclusively polishing agents from the group of silicic acids, aluminum hydroxide, aluminum oxide, dicalcium phosphate dihydrate (CaHPO₄.2H₂O), or mixtures of said friction bodies. These polishing agents may be particularly efficient.

Very particularly preferably, the compositions according to the present specification may contain, based on their weight, 1 to 30 wt % precipitated silicic acid(s) having a specific surface area ≦55 m²/g according to ISO 5794-1, Appendix D. The precipitated silicic acids that exhibit corresponding specific surface areas are preferably used within narrower quantity ranges; particularly preferably, precipitated silicic acids that have even lower specific surface areas according to ISO 5794-1, Appendix D are used. Preferred oral and dental care and cleaning agents according to the present invention contain 2.5 to 25 wt %, preferably 5 to 20 wt %, particularly preferably 7.5 to 17.5 wt %, more preferably 8.0 to 15.0 wt %, and in particular 10.0 to 13.0 wt % precipitated silicic acid(s) having a specific surface area 55 m²/g according to ISO 5794-1, Appendix D.

Particularly preferred oral and dental care and cleaning agents may be characterized in that all precipitated silicic acid(s) contained in the agent have a specific surface area ≦53 m²/g, preferably ≦51 m²/g, more preferably ≦49 m²/g, and in particular ≦47 m²/g according to ISO 5794-1, Appendix D.

In further preferred agents, the precipitated silicic acids used may be characterized by further physical parameters. Precipitated silicic acids that are preferably to be used have tamped densities >360 g/l (measured according to ISO 787-11), particularly preferably >375 g/l, more preferably >400 g/l, and in particular >425 g/l.

It is further preferred to employ precipitated silicic acids that have a DBP absorption according to DIN 53601 of less than 140 g/100 g. Very particularly preferred precipitated silicic acids to be used according to the present invention have a DBP absorption according to DIN 53601 of less than 135 g/100 g, preferably a DBP absorption according to DIN 53601 of less than 130 g/100 g, and in particular less than 125 g/100 g.

Agents particularly preferred may contain 2.5 to 25 wt %, preferably 5 to 20 wt %, particularly preferably 7.5 to 17.5 wt %, more preferably 8.0 to 15.0 wt %, and in particular 10.0 to 14.0 wt % precipitated silicic acid(s) having a specific surface area 45 m²/g according to ISO 5794-1, Appendix D and a tamped density >425 g/l (measured according to ISO 787-11).

Agents further preferred may contain 2.5 to 25 wt %, preferably 5 to 20 wt %, particularly preferably 7.5 to 17.5 wt %, more preferably 8.0 to 15.0 wt %, and in particular 10.0 to 14.0 wt % precipitated silicic acid(s) having a specific surface area 45 m²/g according to ISO 5794-1, Appendix D and a DBP absorption according to DIN 53601 of less than 125 g/100 g.

Agents particularly preferred may contain 2.5 to 25 wt %, preferably 5 to 20 wt %, particularly preferably 7.5 to 17.5 wt %, more preferably 8.0 to 15.0 wt %, and in particular 10.0 to 14.0 wt % precipitated silicic acid(s) having a specific surface area 45 m²/g according to ISO 5794-1, Appendix D and a tamped density >425 g/l (measured according to ISO 787-11) and a DBP absorption according to DIN 53601 of less than 125 g/100 g.

The quantity of polishing agent(s) used may be preferably adapted to the total quantity of surfactant(s). For example, agents may include polishing agent(s) such that the weight ratio of polishing agent(s) to surfactant(s) is ≦6, i.e. the polishing agents may be used at most at a sixfold (weight) excess in terms of the surfactants. The “weight ratio” may refer to the weight ratio of all substances from the groups recited or to the weight ratio of the active substances being calculated. For example, if an agent contains 12 wt % polishing agent and 4 wt % of a 50% surfactant solution, the weight ratio is then 12/2=6. If an agent contains, for example, 12 wt % polishing agent and 4 wt % of a 50% surfactant solution as well as 1 wt % pure sodium dodecyl sulfate, the weight ratio is then 12/(2+1)=4.

In preferred agents the aforementioned weight ratio falls within an even narrower range. Particularly preferred oral and dental care and cleaning agents are characterized in that the weight ratio of polishing agent(s) to surfactant(s) is in the range of ≧4 to ≦5.9, preferably in the range of ≧4.5 to ≦5.8, more preferably in the range of ≧5 to ≦5.7, and in particular in the range of ≧5.25 to ≦5.6.

Oral and dental care and cleaning agents, in particular toothpastes, can also contain, for example, substances that are effective against plaque and/or tartar.

Plaque is a coarse, sticky coating on the teeth that is made up of saliva, bacteria, and food residues. If mineral salts (e.g. calcium, phosphates) from saliva become deposited in the plaque, this causes the formation of hard white or yellowish deposits on the teeth called “tartar.” Plaque can in turn easily become deposited in the porous tartar and attack the gums.

Bacteria on the tooth surface break down carbohydrates from food, particularly sugar, into acid. This acid dissolves the tooth substance, and caries (cavities) occur. The minerals calcium and phosphate are, in particular, dissolved out of the enamel. After the enamel coating, internal layers of the tooth are also attacked. Bacteria can penetrate into the tooth pulp and cause inflammation there. This then usually causes intense toothache.

As already mentioned, plaque contains bacteria, so that antimicrobial substances are suitable for counteracting plaque. They furthermore have an effect as preservatives.

The preservatives sorbic acid (E 200), potassium sorbate (E 202), calcium sorbate (E 203), benzoic acid (E 210), sodium benzoate (E 211), potassium benzoate (E 212), calcium benzoate (E 213), ethyl-4-hydroxybenzoate (E 214), ethyl-4-hydroxybenzoate, sodium salt (E 215), propyl-4-hydroxybenzoate (E 216), propyl-4-hydroxybenzoate, sodium salt (E 217), methyl-4-hydroxybenzoate (E 218), methyl-4-hydroxybenzoate, sodium salt (E 219), sulfur dioxide (sulfurous acid), (E 220), sodium sulfite (E 221), sodium hydrogen sulfite (E 222), sodium disulfite (E 223), potassium disulfite (E 224), calcium sulfite (E 226), calcium hydrogen sulfite (E 227), potassium hydrogen sulfite (E 228), biphenyl (E 230), orthophenylphenol (2-biphenylol), (E 231), sodium orthophenyl phenolate (E 232), Nisin (E 234), natamycin (E 235), formic acid (E 236), sodium formate (E 237), calcium formate (E 238), hexamethylenetetramine (E 239), dimethyl dicarbonate (E 242), potassium nitrite (E 249), sodium nitrite (E 250), sodium nitrate (E 251), acetic acid (E 260), potassium acetate (E 261), sodium acetate (E 262), calcium acetate (E 263), lactic acid (E 270), propionic acid (E 280), sodium propionate (E 281), calcium propionate (E 282), potassium propionate (E 283), boric acid (E 284), sodium tetraborate (E 285), hydroxysuccinic acid (malic acid), (E 296), fumaric acid (E 297), lysozyme (E 1105), which are also permitted in food, can be employed, for example, in oral and dental care and cleaning agents.

Preferred substances may be selected from alkylpyridinium salts, in particular cetylpyridinium chloride, p-hydroxybenzoic acid methyl, ethyl, or propyl esters, sodium sorbate, sodium benzoate, bromochlorophen, triclosan, phenyl salicylic acid esters, biguanides, e.g. chlorhexidine (1,1′-hexamethylene bis-[5-(4-chlorophenyl)]biguanide), thymol, etc.

Oral and dental care and cleaning agents preferred may be characterized in that that they additionally contain anti-plaque active agents, preferably alkylpyridinium salts, in particular cetylpyridinium chloride, p-hydroxybenzoic acid methyl, ethyl, or propyl esters, sodium sorbate, sodium benzoate, bromochlorophen, triclosan, hexetidine, phenyl salicylic acid esters, biguanides, e.g. chlorhexidine, thymol, preferably in quantities from 0.05 to 5 wt %, preferably from 0.10 to 2.5 wt %, and in particular from 0.30 to 1.5 wt %, based in each case on the total agent.

The quantities indicated refer to the total quantity of anti-plaque active agents, individual active agents preferably being employed in narrower quantity ranges. Agents preferred according to the present invention contain, for example, 0.1 to 2 wt %, preferably 0.2 to 1.75 wt %, more preferably 0.3 to 1.5 wt %, and in particular 0.4 to 1.0 wt % sodium benzoate. Agents further preferred may also be characterized in that they contain 0.005 to 0.1 wt %, preferably 0.01 to 0.075 wt %, and in particular 0.02 to 0.05 wt % chlorhexidine. Chlorhexidine is preferably used together with alkylpolyglycosides (APG); alkylglycosides having 8 to 18 carbon atoms in the alkyl group and an average degree of oligomerization of the glycoside residue from 1 to 3 are contained in a quantity from 0.025 to 2.5 wt % in preferred oral and dental care and cleaning agents.

Substances effective against tartar can be, for example, chelating agents such as e.g. ethylenediaminetetraacetic acid and sodium salts thereof, pyrophosphate salts such as water-soluble dialkali or tetraalkali metal pyrophosphate salts, e.g. Na₄P₂O₇, K₄P₂O₇, Na₂K₂P₂O₇, Na₂H₂P₂O₇, and K₂H₂P₂O₇, or polyphosphate salts that can be selected, for example, from water-soluble alkali metal tripolyphosphates such as sodium tripolyphosphate and potassium tripolyphosphate.

Oral and dental care and cleaning agents preferred may be characterized in that they additionally contain phosphate(s), preferably alkali metal phosphate(s), and in particular sodium tripolyphosphate, preferably in quantities from 1 to 10 wt %, particularly preferably from 2 to 8 wt %, and in particular from 3 to 7 wt %, based in each case on the total agent.

Natural and/or synthetic water-soluble polymers such as alginates, carrageenates, tragacanth, starch and starch ethers, cellulose ethers such as carboxymethyl cellulose (sodium salt), hydroxyethyl cellulose, methylhydroxypropyl cellulose, guar, acacia gum, agar-agar, xanthan gum, succinoglycan gum, locust bean flour, pectins, water-soluble carboxyvinyl polymers (e.g. Carbopol® grades), polyvinyl alcohol, polyvinylpyrrolidone, polyethylene glycols, in particular those having molecular weights from 1500 to 1,000,000, serve, for example, as consistency regulators (respectively. binding agents).

Further substances that are suitable for viscosity control are, for example, sheet silicates such as montmorillonite clays, colloidal thickening silicic acids such as aerogel silicic acids, pyrogenic silicic acids, or ultra-finely ground precipitated silicic acids. Viscosity-stabilizing additives from the group of cationic, zwitterionic, or ampholytic nitrogen-containing surfactants, hydroxypropyl-substituted hydrocolloids, or polyethylene glycol/polypropylene glycol copolymers having an average molecular weight from 1000 to 5000, or a combination of the aforesaid compounds, can also be used in toothpastes.

The agents in particular toothpastes, may also contain substances to increase mineralizing potential, for example calcium-containing substances such as calcium chloride, calcium acetate, and dicalcium phosphate dihydrate. The concentration of calcium-containing substance depends on the solubility of the substance and on the interaction with other substances contained in the oral and dental care agent.

Besides the components recited, the oral and dental care and cleaning agents may contain further adjuvants and additives. One additive that is a toothpaste component that is particularly effective in the dental care agents is calcium glycerophosphate, which is the calcium salt of glycerol-1-phosphoric acid or of glycerol-2-phosphoric acid or of glycerol-3-phosphoric acid that is enantiomeric to glycerol-1-phosphoric acid, or of a mixture of said acids. The compound has a remineralizing effect in the oral and dental care and cleaning agent as it supplies both calcium ions and phosphate ions. Calcium glycerophosphate is contained in the oral and dental care and cleaning agent preferably in quantities from 0.01 to 1 wt %. The oral and dental care and cleaning agent may contain adjuvants and additives in quantities in total of up to 10 wt %.

Oral and dental care and cleaning agents may, with particular preference, contain anti-caries active agents. These may be selected, for example, from organic or inorganic fluorides, e.g. from sodium fluoride, potassium fluoride, sodium monofluorophosphate, and sodium fluorosilicate. Zinc fluoride and tin(II) fluoride are also preferred. A quantity from 0.01 to 0.2 wt % fluoride, in the form of the aforesaid compounds, should preferably be contained.

Oral and dental care and cleaning agents according may contain anti-caries active agents, preferably fluorine compound(s), in particular sodium fluoride, potassium fluoride, sodium monofluorophosphate, zinc fluoride, tin fluoride, and sodium fluorosilicate, preferably in quantities from 0.01 to 5 wt %, preferably from 0.1 to 2.5 wt %, and in particular from 0.2 to 1.1 wt %, based in each case on the total agent, are preferred according to the present invention.

The dental care agents may be improved in terms of their organoleptic properties, for example, by adding flavor oils and sweetening agents.

All natural and synthetic flavors usual for oral and dental care agents can be used as flavor oils. Natural flavors can be contained both in the form of natural essential oils isolated from botanicals, and as the individual components isolated therefrom.

Suitable flavors are, for example, peppermint oil, spearmint oil, eucalyptus oil, anise oil, fennel oil, caraway oil, menthyl acetate, cinnamaldehyde, anethol, vanillin, thymol, and mixtures of said components.

Suitable sweetening agents are, for example, sodium saccharin, sodium cyclamate, sucrose, lactose, Meltose, fructose, as well as maltose and dextrose.

A further subject of the present disclosure is the use of agents for cleaning teeth by means of electric toothbrushes.

A further subject of the present disclosure is a method for cleaning teeth, characterized in that an agent is applied onto the brush head of an electric toothbrush and the teeth are brushed with the electric toothbrush.

A further subject of the present invention is a method for tooth cleaning, characterized by the steps of
a) providing a toothbrush whose brush head can be set in motion electrically,
b) applying 0.5 to 5 g of an agent onto the brush head,
c) brushing teeth for 30 to 300 seconds with the agent, employing the brush head set in motion electrically.

The statements made regarding the agents apply mutatis mutandis with respect to preferred examples of the use according to the present disclosure and of the methods according to the present disclosure.

The following dentifrices were manufactured and were used under identical conditions on an electric toothbrush. Quantities of the dentifrices are indicated in %.

| | V1 | V2 | E |
|---|---|---|---|
| Sorbitol 70% | 18.5 | 18.5 | 18.5 |
| Glycerol | 22.5 | 22.5 | 22.5 |
| Silicic acid, abrasive | 12 | 12 | 12 |
| Silicic acid, thickening | 9 | 9 | 9 |
| Cl 42090 | 0.005 | 0.005 | 0.005 |
| Silicic acid particles | 1.5 | 1.5 | 1.5 |
| Disodium phosphate | 0.2 | 0.2 | 0.2 |
| Trisodium phosphate | 0.25 | 0.25 | 0.3 |
| Sodium fluoride | 0.32 | 0.32 | 0.32 |
| Saccharin | 0.25 | 0.25 | 0.25 |
| Zinc sulfate 7 H2O | 0.09 | 0.09 | 0.09 |
| Xanthan FN | 0.9 | 0.9 | 0.9 |
| Cocamidopropyl Betaine | 0 | 1 | 0.2 |
| Sodium lauryl sulfate | 2.2 | 1.2 | 2 |
| Flavor | 1 | 1 | 1 |
| Water | to 100 | to 100 | to 100 |

Formula V1 contained no amphoteric surfactant; in formula V2 the weight ratio of anionic to amphoteric surfactant(s) was 1.2; and in formula E the weight ratio of anionic to amphoteric surfactant(s) was 10 (corresponding to ≧8).

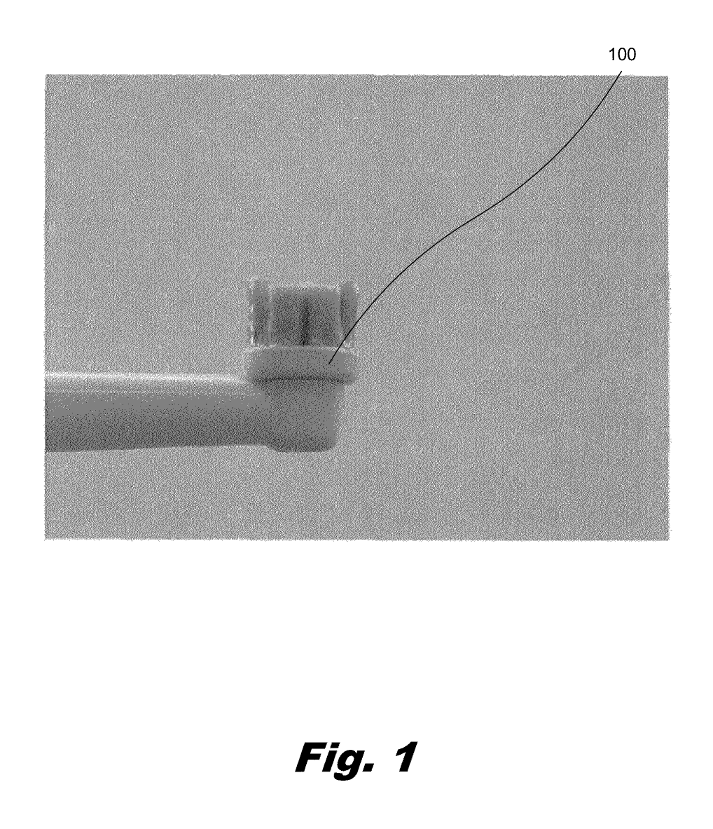

The compositions were applied using an electric toothbrush. Photographic images of the brush heads were then prepared as indicated in FIGS. 1-3. Parameters for utilization on electric toothbrush:

1 g toothpaste

brushing time: 3 min

brushing substrate: undulating plate.

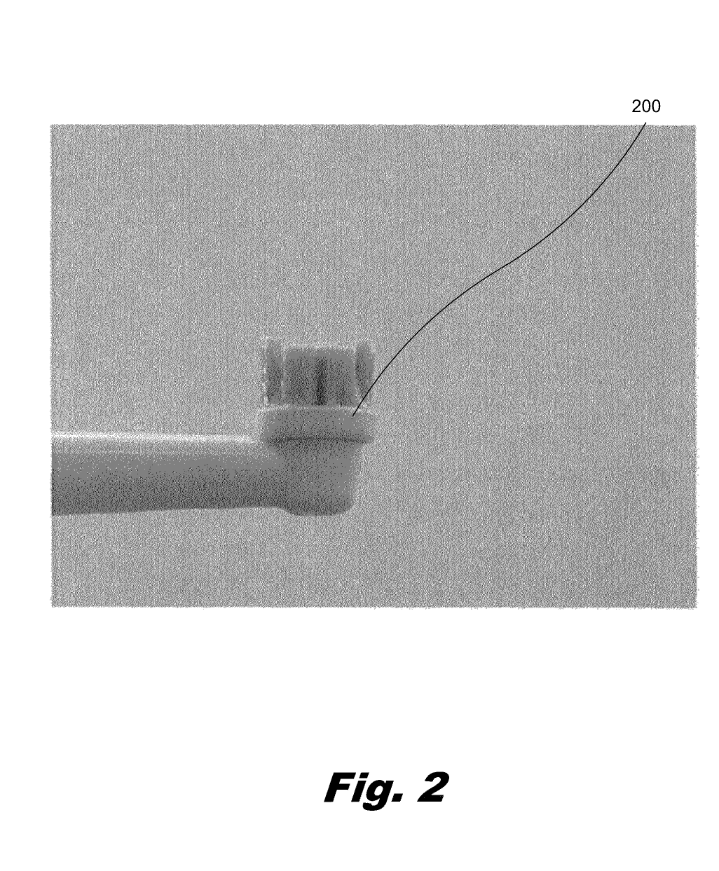

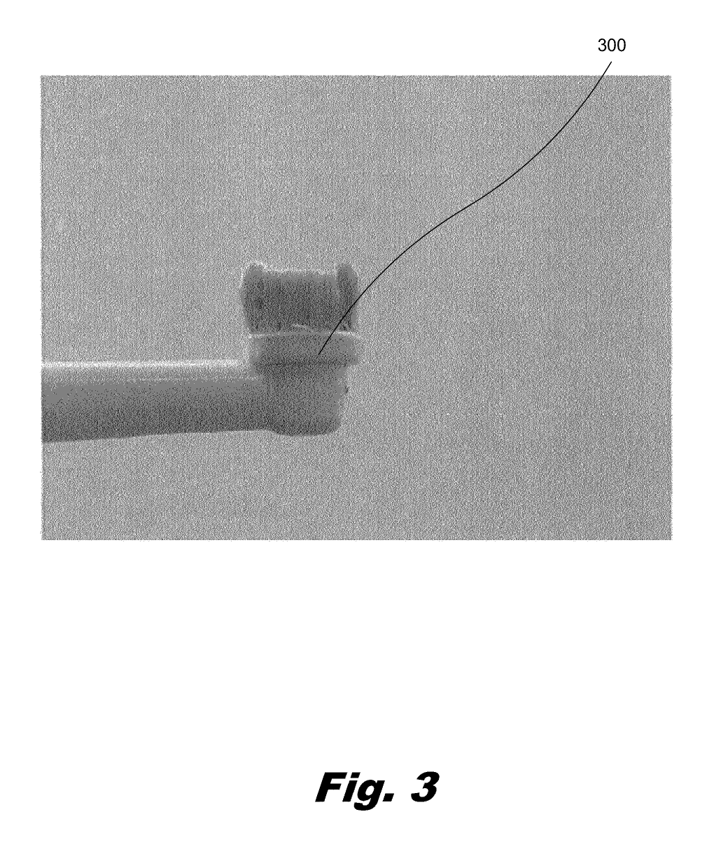

In addition, in the case of compositions V1 and V2, the brush heads of the electric toothbrush exhibited substantial incrustations after 100 cycles each (rinsed under running water between cycles). After 200 cycles the mechanism in the brush heads of the electric toothbrush was greatly impaired in the case of compositions V1 and V2, whereas with composition E it functioned without problems. As indicated above, FIG. 1 indicates a brush head (100) utilized with a dentifrice according to formula V1. FIG. 2 indicates a brush head (200) utilized with a dentifrice according to formula V2. FIG. 3 indicates a brush head (300) utilized with a dentifrice according to formula E. As indicated in FIGS. 1-3, the brush heads (100, 200) utilized with formula V1 and V2 dentifrice, respectively, exhibited pooper foaming characteristics as compared to the brush head (300) utilized with a formula E dentifrice.

While at least one example has been presented in the foregoing detailed description, it should be appreciated that a vast number of variations exist. It should also be appreciated that the examples are only examples, and are not intended to limit the scope, applicability, or configuration of the specification in any way. Rather, the foregoing detailed description will provide those skilled in the art with a convenient road map for implementing an example of the present specification, it being understood that various changes may be made in the function and arrangement of elements described in an examples without departing from the scope of the specification as set forth in the appended claims and their legal equivalents.

What is claimed is:

1. An oral and dental care and cleaning agent comprising:
at least one anionic surfactant; and
at least one amphoteric surfactant;
in which a weight ratio of the at least one anionic surfactant to the at least one amphoteric surfactant is greater than or equal to 6.

2. The oral and dental care and cleaning agent of claim 1, in which the weight ratio of the at least one anionic surfactant to the at least one amphoteric surfactant is greater than or equal to 6.5.

3. The oral and dental care and cleaning agent of claim 1, in which the at least one anionic surfactant comprises between 0.25 and 4 weight percent of the oral and dental care and cleaning agent.

4. The oral and dental care and cleaning agent of claim 1, in which the at least one amphoteric surfactant comprises between 0.01 and 2.0 weight percent of the oral and dental care and cleaning agent.

5. The oral and dental care and cleaning agent of claim 1, in which a combined quantity of the at least one anionic surfactant and the at least one amphoteric surfactant comprises between 0.5 and 5.0 weight percent of the oral and dental care and cleaning agent.

6. The oral and dental care and cleaning agent of claim 1, in which the weight ratio of the at least one anionic to amphoteric surfactant(s) is greater than or equal to 10.25 and less than or equal to 20.

7. The oral and dental care and cleaning agent of claim 1, further comprising an abrasive, a silicic acid, aluminum hydroxide, aluminum oxide, calcium pyrophosphate, chalk, dicalcium phosphate dihydrate (CaHPO₄.2H₂O), sodium aluminum silicates, in particular zeolite A, organic polymers, polymethacrylates, or combinations thereof in quantities between 1 and 30 wt % of the oral and dental care and cleaning agent.

8. The oral and dental care and cleaning agent of claim 1, further comprising a polishing agent, wherein a weight ratio of the polishing agent to the surfactants is greater than or equal to 4% and less than or equal to 5.9%.

9. The oral and dental care and cleaning agent of claim 1, in which the oral and dental care and cleaning agent is configured for cleaning teeth by means of electric toothbrushes.

10. A method for cleaning teeth, the method comprising:
applying an oral and dental care and cleaning agent onto the brush head of an electric toothbrush, in which the oral and dental care and cleaning agent comprises:
at least one anionic surfactant; and
at least one amphoteric surfactant;
in which a weight ratio of the at least one anionic surfactant to the at least one amphoteric surfactant is at least 6:1; and
brushing the teeth with the electric toothbrush.

11. A method for cleaning teeth, the method comprising:
providing a toothbrush whose brush head can be set in motion electrically,
applying 0.5 to 5 g of an agent onto the brush head, in which the agent comprises:
at least one anionic surfactant; and
at least one amphoteric surfactant;
in which a weight ratio of the at least one anionic surfactant to the at least one amphoteric surfactant is at least 6:1;
brushing teeth, with the toothbrush, for 30 to 300 seconds with the agent.
